(19) United States
(12) Patent Application Publication
(10) Pub. No.: US 2003/0223944 A1
(43) Pub. Date: Dec. 4, 2003

(54) Metallic coating process for giving keratin fibres shampoo-remanent cosmetic properties

(76) Inventors: Aude Livoreil, Paris (FR); Gabin Vic, Compiegne (FR); Franck Giroud, Clichy (FR)

Correspondence Address: NIXON & VANDERHYE, PC, 1100 N GLEBE ROAD, 8TH FLOOR, ARLINGTON, VA, 22201-4714, US

(21) Appl. No.: 10/407,911

(22) Filed: Apr. 7, 2003

Related U.S. Application Data
(60) Provisional application No. 60/372,455, filed on Apr. 16, 2002.

(30) Foreign Application Priority Data
Apr. 8, 2002 (FR) 0204352

(51) Int. Cl. A61K 7/075; A61K 7/06

(52) U.S. Cl. 424/70.1; 510/119

(57) ABSTRACT

The present invention relates to a cosmetic treatment process for giving keratin fibres shampoo-remanent cosmetic properties, which consists in reducing, in a cosmetically acceptable medium, at least one metal salt in the presence of at least one reducing agent, directly on the keratin fibre to form the corresponding native metal in situ.

The present invention relates to a metallic coating process for giving keratin fibres, and especially the hair, shampoo-remanent cosmetic properties.

It is known practice to treat keratin fibres by physicochemical adsorption or deposition of material onto the surface of the fibres in order to increase their sheen, to colour them or to make them more heat-resistant, more lightfast, etc.

Patent application EP-A-1 064 918 describes the application to the hair of a cosmetic composition containing particles of metallic type.

Patent application JP-A-03 090 012 describes the application to the skin and the hair of a cosmetic composition comprising proteins tinted with gold particles.

Patent application JP-A-03 077 806 describes the application to the skin and the hair of a composition comprising a powder of silk fibres tinted with gold particles.

Patent application JP-A-03 077 806 describes the application to the skin and the hair of a cosmetic composition comprising a hydrosol of fine gold particles.

However, these treatments have relatively poor shampoo-remanent effects since the material deposited is rapidly removed by the action of water and surfactants. The standard processes of cosmetic hair treatment thus give the deposit little remanence under the action of shampoos and the cosmetic effects provided are very quickly eroded.

The present invention proposes to overcome these drawbacks.

In particular, the Applicant has just discovered, surprisingly, that coating keratin fibres with a native metal gives the keratin fibres shampoo-remanent cosmetic properties.

Thus, the keratin fibres bearing this metallic coating have shampoo-remanent cosmetic properties, for instance sheen, glints or coloration.

One subject of the present invention is thus a cosmetic treatment process for giving keratin fibres shampoo-remanent cosmetic properties, which consists in forming in situ on the fibre a native metal by reducing a metal salt in the presence of a reducing agent.

Other subjects of the invention will become apparent on reading the description and the examples that follow.

The process in accordance with the invention is essentially characterized in that it consists in reducing, in a cosmetically acceptable medium, at least one metal salt in the presence of at least one reducing agent, directly on the keratin fibre to form the corresponding native metal in situ.

According to one embodiment of the invention, the keratin fibre is reduced beforehand, prior to being placed in contact with the metal salt, so as to release the SH groups which have the property of reacting with metals to form an S-metal bond which increases the remanence of the metal in the fibre.

In a concomitant or, preferably, subsequent step, active agents chosen from thiols, thiolates, disulphides, thioethers, xanthates, thiocarbamates, thiosulphates and thiolactones, provided that they do not reduce the metal salts used in the above process, may be grafted onto the keratin fibre coated with a metallic deposit obtained according to the process described above.

The metal of the metal salt may be chosen from aluminium, alkali metals, alkaline-earth metals, transition metals, rare-earth metals and alloys thereof. Preferably, the metal is chosen from aluminium, copper, silver, gold, indium, iron, platinum, nickel, molybdenum, titanium, tungsten, antimony, palladium, zinc and tin, and alloys thereof.

The metal salt may be chosen from mineral or organic, acidic or basic salts such as nitrates, sulphates, carbonates, halides, bromates, phosphates and sulphonates.

Preferably, the metal salt is chosen from AgNO₃, AgBrO₃, Ag₂SO₄, AgBr, Ag₂CO₃, AgCl, AgF, AgI, HAuCl₄, AuBr₃, AuCl₃, K₂PdCl₄, CuCl, CuBr, CuSO₄ and Na₂PtCl₆. The salts of noble metals, for instance HAuCl₄, AgNO₃, K₂PdCl₄ and Na₂PtCl₆, are preferred. Mixtures of metal salts may also be used.

The reducing agent may be chosen from organic or mineral reducing agents such as NaBH₄, KBH₄, BH₃, NADPH, NADH, H₂, 9-BBN (9-borabicyclo[3.3.1]nonane), LiAlH₄, NaAlEt₂H₂, Na₂S₂O₃, diisoamylborane, ascorbic acid and cyclic or aliphatic compounds containing the enediol chemical group —(OH)C═C(OH)— including the isomeric forms, salts thereof and esters thereof, hydroquinone, benzoquinone, NaBH(OAc)₃, formamidinesulphinic acid (FASA), N-phenylformamidinesulphinic acid, phosphines, NaBH₃CN, Na₂S₂O₄, BMS (bismercaptoethyl sulphone), DTT (dithiothreitol), DMH (N,N′-dimethyl-N,N′-bis(mercapto-acetyl)hydrazine), thiols, thiolates, citrates, sulphites, bisulphites, monopersulphates and bisulphates of alkali metals, enzymes (such as, for example, the protein disulphide isomerase or thioredoxine), and sulphinates, in particular hydroxymethanesulphinates.

The metal salts and the reducing agents are generally used, independently of each other, at a concentration of between 0.0001% and 50%, preferably between 0.01% and 10% and more preferably between 0.05% and 5%, relative to the cosmetically acceptable medium.

The cosmetically acceptable medium used in accordance with the invention preferably consists of water, optionally mixed with an organic solvent chosen from the group comprising C₁-C₄ alcohols such as ethanol or isopropanol, C₅-C₁₀ alkanes, acetone, methyl ethyl ketone, methyl acetate, butyl acetate, ethyl acetate, dimethoxyethane and diethoxyethane, and mixtures thereof.

The compositions used according to the invention comprising at least one metal salt or at least one reducing agent in a cosmetically acceptable medium may also comprise common cosmetic additives chosen especially from adhesives, thickeners, softeners, antifoams, sunscreens, antiperspirants, acidifying agents, moisturizers, basifying agents, colorants, pigments, fragrances, preserving agents, anionic, cationic, nonionic or amphoteric surfactants, fixing polymers, especially nonionic, cationic or amphoteric fixing polymers, non-fixing polymers, volatile or non-volatile silicones, plant, animal, mineral or synthetic oils, proteins, vitamins and polyols, and mixtures thereof.

The compositions used in accordance with the invention may be packaged in various forms, especially in an aerosol device, with a propellant. They may be used in rinse-out or leave-in application.

The propellant is preferably chosen from air, carbon dioxide, compressed nitrogen, dimethyl ether, C₃-C₅ alkanes, for instance n-butane, propane or isobutane, halohydrocarbons, for instance 1,1-difluoroethane, mixtures of dimethyl ether and of C₃-C₅ alkanes, and mixtures of 1,1-difluoroethane and of dimethyl ether and/or of C₃-C₅ alkanes.

The process in accordance with the invention may be used for fixing and/or holding the hair, haircare, shampooing or conditioning the hair, for example to give it softness, or alternatively for making up the hair.

The compositions used according to the invention may be in various presentation forms, such as a lotion, a gel, a stick, a cream, a simple or multiple emulsion, a spray, a mousse, a shampoo or a conditioner.

The process for treating keratin fibres, especially the hair, according to the invention generally comprises the following steps:

a composition containing at least one metal salt, in a cosmetically acceptable medium, is applied to the keratin fibres,

after an action time ranging from 30 seconds to 30 minutes and preferably from 1 to 15 minutes, with or without the action of heat, and optional rinsing, a composition containing at least one reducing agent, in a cosmetically acceptable medium, is applied,

after a further action time ranging from 30 seconds to 30 minutes and preferably from 1 to 15 minutes, with or without the action of heat, the fibres are rinsed.

The metal salt and the reducing agent may be chosen from those described above.

It should be noted that the metallic coating according to the invention may be removed from the keratin fibres, if need be, by simple treatment with aqueous hydrogen peroxide solution at acidic pH at a concentration of between 1 and 30 volumes (between 0.3 and 9% by weight).

The keratin fibres may be coated beforehand with a polymer that has affinity for the surface, for instance polyamines (PEI or polylysines), polysaccharide derivatives (hydroxypropylcellulose, hydroxypropyl guar or Jaguar) or may have undergone a standard cosmetic treatment, for instance an oxidation-dyeing, permanent-waving or bleaching operation, shampooing or an application of styling product.

According to one embodiment of the invention, the keratin fibres may be reduced, before applying the composition containing at least one metal salt. This reduction may be performed with one of the reducing agents described above and in particular the reducing agents used in the context of permanent-waving. One of the preferred reducing agents is thioglycolic acid. The fibres may then be rinsed. The metal salt(s) and then the reducing agent(s) are then applied as described above.

It is also possible to apply to the keratin fibres, concomitantly with the metallic coating or, preferably, subsequently, a composition containing a compound chosen from thiols, thiolates, disulphides, thioethers, xanthates, thiocarbamates, thiosulphates and thiolactones. These compounds may bear substituents with cosmetic activity. These substituents may originate, for example, from natural or synthetic, water-soluble or water-insoluble polymers, organic or metallic particles, pigments, sunscreens, antioxidants, colorants or conditioners.

The examples that follow are intended to illustrate the invention.

### EXAMPLE 1

### Silver Metallic Coating on Non-Reduced Pigmented Natural Hair, Grey Hair or Slightly Lightened Hair

The locks (1 g) are washed with shampoo, dried for 20 minutes at 65° C. and each placed in a suitable drainer. 2 ml of aqueous silver nitrate solution (0.46 M) are applied to each lock and left to act for 5 minutes. 1 ml of aqueous sodium borohydride solution (0.92 M) is then applied to each lock and is left to act for 15 minutes. The locks are then rinsed with water and dried for 20 minutes at 60° C.

The following visual results are obtained:

the locks of pigmented natural hair are blacker, with metallized shiny glints;

the locks of grey hair are dyed golden-yellow with metallized shiny glints;

the locks of slightly lightened sensitized hair are dyed coppery with metallized shiny glints.

The locks are washed ten times with a standard shampoo and no appreciable difference in coloration is observed between an unwashed lock and a lock washed ten times. The colour obtained is thus shampoo-remanent.

The presence of a silver coating on the surface of the hair was confirmed by scanning electron microscopy and EDX analysis.

### EXAMPLE 2

### Metallic Silver Coating on Reduced Pigmented Natural Hair, Grey Hair or Slightly Lightened Hair

Step 1: Reduction of the Locks

The locks (1 g) are each placed in a suitable drainer. 2.4 ml of aqueous thioglycolic acid solution (1 M, pH 9) are applied to each lock and left to act for 15 minutes. The locks are then washed with water and dried for 20 minutes at 60° C. and used immediately.

Step 2: Treatment with Silver Nitrate and Reduction

The reduced locks (1 g) are each placed in a suitable drainer. For each lock, 2 ml of aqueous silver nitrate solution (0.46 M) are applied and left to act for 5 minutes. 1 ml of aqueous sodium borohydride solution (0.92 M) is then applied to each lock and left to act for 15 minutes. The locks are then rinsed with water and dried for 20 minutes at 60° C.

The same visual results as in Example 1 are obtained.

### EXAMPLE 3

### Removal of the Metallic Silver Coating

A lock of slightly lightened hair, treated according to Example 1, is soaked in 30-volumes aqueous hydrogen peroxide solution (9% by weight). The coppery shade of the lock disappears instantaneously and the lock regains its original colour with a strong evolution of gas. By virtue of this treatment, the metallic coating present on the hair is thus removed.

### EXAMPLE 4

### Grafting of 1-Octadecanethiol to Locks of Sensitized Hair Coated with a Metallic Silver Coating

The locks (1 g) treated according to Example 1 are each placed in a suitable drainer. 5 ml of aqueous NaOH solution at 1 mM and then 5 ml of a thiol solution at 0.2 mM are applied to each lock. The thiol solution is obtained by dissolving 0.654 g of 1-octadecanethiol in ethanol (1 litre) and this solution is then diluted 11-fold in ethanol. The locks are incubated overnight at room temperature. The locks are subsequently drained and then rinsed with 20 ml of 50/50 EtOH/1 mM NaOH_{aq} solution (v/v). Finally, the locks are rinsed with water and dried for 20 minutes at 60° C., and then washed 5 times with a standard shampoo.

In order to compare the locks treated with 1-octadecanethiol with the untreated locks coated according to Example 1, a sensory test is used.

Feel quality (from 0 to 5, 0=very poor, 5=very good)

Softness (from 0 to 5, 0=very poor, 5=very good)

Cleanliness of the fingers after touching (from 0 to 5, 0=very poor, 5=very good)

The results are collected in the following table:

| | | | Cleanliness of |
|---|---|---|---|
| Locks | Feel quality | Softness | the fingers |
| Treated locks | 4 | 4.5 | 4 |
| Untreated locks | 3 | 2.5 | 4 |
| (according to | | | |
| Example 1) | | | |

The locks treated with 1-octadecanethiol and washed thus have a feel quality and a softness far superior to the untreated locks according to Example 1.

1. Cosmetic treatment process for giving keratin fibres shampoo-remanent cosmetic properties, characterized in that it consists in reducing, in a cosmetically acceptable medium, at least one metal salt in the presence of at least one reducing agent, directly on the keratin fibre to form the corresponding native metal in situ.

2. Process according to claim 1, characterized in that the metal of the metal salt is chosen from aluminium, alkali metals, alkaline-earth metals, transition metals, rare-earth metals and alloys thereof.

3. Process according to claim 1 or 2, characterized in that the metal is chosen from aluminium, copper, silver, gold, indium, iron, platinum, nickel, molybdenum, titanium, tungsten, antimony, palladium, zinc and tin, and alloys thereof.

4. Process according to any one of the preceding claims, characterized in that the metal salt is an organic or mineral, acidic or basic salt.

5. Process according to any one of the preceding claims, characterized in that the metal salt is chosen from nitrates, sulphates, carbonates, halides, bromates, phosphates and sulphonates.

6. Process according to any one of the preceding claims, characterized in that the metal salt is chosen from AgNO₃, AgBrO₃, Ag₂SO₄, AgBr, Ag₂CO₃, AgCl, AgF, AgI, HAuCl₄, AuBr₃, AuCl₃, K₂PdCl₄, CuCl, CuBr, CuSO₄ and Na₂PtCl₆, and mixtures thereof.

7. Process according to any one of the preceding claims, characterized in that the metal salt is chosen from HAuCl₄, AgNO₃, K₂PdCl₄ and Na₂PtCl₆, and mixtures thereof.

8. Process according to any one of the preceding claims, characterized in that the reducing agent is chosen from mineral or organic reducing agents.

9. Process according to any one of the preceding claims, characterized in that the reducing agent is chosen from NaBH₄, KBH₄, BH₃, NADPH, NADH, H₂, 9-BBN (9-borabicyclo[3.3.1]nonane), LiAlH₄, NaAlEt₂H₂, Na₂S₂O₃, diisoamylborane, ascorbic acid and cyclic or aliphatic compounds containing the enediol chemical group —(OH)C═C(OH)— including the isomeric forms, salts thereof and esters thereof, hydroquinone, benzoquinone, NaBH(OAc)₃, formamidinesulphinic acid (FASA), N-phenylformamidinesulphinic acid, phosphines, NaBH₃CN, Na₂S₂O₄, BMS (bismercaptoethyl sulphone), DTT (dithiothreitol), DMH (N,N′-dimethyl-N,N′-bis(mercaptoacetyl)hydrazine), thiols, thiolates, citrates, sulphites, bisulphites, monopersulphates and bisulphates of alkali metals, enzymes (such as, for example, the protein disulphide isomerase or thioredoxine), and sulphinates.

10. Process according to any one of the preceding claims, characterized in that the metal salt(s) and the reducing agent(s) are present at a concentration of between 0.0001% and 50%, preferably between 0.01% and 10% and more preferably between 0.05% and 5%, relative to the cosmetically acceptable medium.

11. Process according to any one of the preceding claims, characterized in that the cosmetically acceptable medium consists of water and/or an organic solvent chosen from the group comprising C₁-C₄ alcohols such as ethanol or isopropanol, C₅-C₁₀ alkanes, acetone, methyl ethyl ketone, methyl acetate, butyl acetate, ethyl acetate, dimethoxyethane and diethoxyethane, and mixtures thereof.

12. Process according to any one of the preceding claims, characterized in that the cosmetically acceptable medium also comprises common cosmetic additives chosen especially from adhesives, thickeners, softeners, antifoams, sunscreens, antiperspirants, acidifying agents, moisturizers, basifyiing agents, colorants, pigments, fragrances, preserving agents, anionic, cationic, nonionic or amphoteric surfactants, fixing polymers, especially nonionic, cationic or amphoteric fixing polymers, non-fixing polymers, volatile or non-volatile silicones, plant, animal, mineral or synthetic oils, proteins, vitamins and polyols, and mixtures thereof.

13. Process according to any one of the preceding claims, characterized in that:
a composition containing at least one metal salt, in a cosmetically acceptable medium, is applied to the keratin fibres,
after an action time ranging from 30 seconds to 30 minutes, and optional rinsing, a composition containing at least one reducing agent, in a cosmetically acceptable medium, is applied under the optional action of heat,
after a further action time of 30 seconds to 30 minutes, under the optional action of heat, the fibres are rinsed.

14. Process according to claim 13, characterized in that, prior to applying the composition containing at least one metal salt to the keratin fibres, the keratin fibres are reduced using a reducing agent as defined in claim 9.

15. Process according to claim 14, characterized in that the prior reduction is performed with thioglycolic acid.

16. Process according to claim 13, characterized in that, prior to applying the composition containing at least one metal salt to the keratin fibres, the keratin fibres are coated with a polymer that has affinity for the surface, for instance polyamines or polysaccharide derivatives.

17. Process according to claim 13, characterized in that, prior to applying the composition containing at least one metal salt to the keratin fibres, the keratin fibres are subjected to a standard cosmetic treatment, for instance an oxidation-dyeing, permanent-wave or bleaching operation, shampooing or an application of styling product.

18. Process according to any one of claims 1 to 17, characterized in that, subsequent to the metallic coating operation or optionally concomitantly, a composition containing at least one compound chosen from thiols, thiolates, disulphides, thioethers, xanthates, thiocarbamates, thiosulphates and thiolactones is applied to the keratin fibres.
